(19) United States
(12) Patent Application Publication
(10) Pub. No.: US 2015/0245938 A1
(43) Pub. Date: Sep. 3, 2015

(54) HEALTH ORTHOSIS FOR FOOT

(71) Applicant: BELSHAN CO., LTD., Tokyo (JP)

(72) Inventor: Minoru HONDA, Tokyo (JP)

(21) Appl. No.: 14/611,723

(22) Filed: Feb. 2, 2015

(30) Foreign Application Priority Data
Feb. 28, 2014 (JP) 2014-038215

(51) Int. Cl. A61F 5/01; A61H 3/00

(52) U.S. Cl. CPC A61F 5/019; A61F 5/0127; A61H 3/00

(57) ABSTRACT

A foot health orthosis to be fitted with a toe includes a flexible ring-like retainer which can be fitted with the toe, and a cushion portion formed on a part of the retainer, which is thicker than the retainer and allowed to be held on a reverse surface of the toe in the fitted state.

### TECHNICAL FIELD

The present invention relates to a foot health orthosis, which is fitted with a toe of one's foot for naturally enhancing the kicking force of the foot during walking so as to prevent deterioration in the muscle of the foot and the body part therearound, and is effective for prevention and correction of abnormality in the toe, for example, floating foot, broad foot, hallux valgus and the like.

### BACKGROUND OF ART

Walking plays important roles in the health maintenance by strengthening muscles of feet and the part therearound and improving blood circulation in addition to the exercise from the functional aspect.

Blood is supplied to every possible parts of the human body by repeating a series of circular motions through the heart serving as the pump for supplementing oxygen and nutritive substances required for cell division, collecting the carbon dioxide and waste product, and returning to the heart again.

The feet are apart from the heart, and likely to be affected by the load gravity. The pressure derived from pumping action of the heart is insufficient to return the blood to the heart, which may require aiding in the circulation by walking.

The muscle of the lower half of the human body occupies ⅔ of the whole muscle, which is integrated with capillaries of venous blood. Through the walking motion, the foot serves to expand and contract the muscles of the foot, calf, thigh and the like repeatedly so that the blood flowing through the foot and the leg is pumped up toward the vein with the valve that prevents the backflow of blood. This motion is called “milking action”, which realizes the pumping action similar to that of the heart. This is why the “foot” is referred to as “the second heart”.

Furthermore, the muscle, blood vessel and nerve of the foot are closely linked with the brain and various internal organs, which may be stimulated by walking, thus promoting health physically and mentally.

It is essential to pay attention to the walking balance from landing to kicking with the foot for the purpose of enhancing the effect of walking. The toes perform an important function to achieve the walking balance and kicking.

The walker with normal soles of the feet walks on the whole soles including (1) heel, (2) each root of toes, and (3) tiptoe. Meanwhile, the walker with weak kicking force will walk only on two points of the sole, that is, (1) heel and (2) each root of toes while having the gravity shifted toward the heel, which may be referred to as the sliding feet. In the aforementioned case, the walker is likely to take the posture that hardly requires use of the toes while walking. As a result, the muscle of the subject part is weakened, leading to the state where the toes are lifted, which is referred to as the floating toes.

The floating toes exhibit insufficient fast-standing force to make the walking balance unstable. Excessive burden may be imposed on the feet, lower limb, neck and the like to keep the walking balance with the upper part, thus causing various symptoms such as pain in various parts of the body.

The foot of the human who is walking on two legs has three arches formed on the sole to allow the human to support the whole weight, and concentrate and further withstand walking and running motions to be described as follows (See FIG. 7).

(1) the arch between the heel and root of a hallux (big toe): lateral arch
(2) the arch between the heel and root of a microdactyly (little toe): medial arch
(3) the arch between the root of the hallux (big toe) and root of the microdactyly (little toe): metatarsal arch

Each of those arches is formed of the muscle and ligament, playing important roles as follows:

Applying kicking force to foot (spring function);

Absorbing shock and load; and

Protecting muscle, blood vessel, nerve of sole.

Deterioration in the metatarsal arch may open the metatarsal like a fan, which is referred to as “broad foot” as it is laterally expanded, thus causing the hallux valgus and digitus minimus varus. The spring force for allowing the foot to kick is weakened to lose the balance of the body posture, which may increase the load exerted to the feet and the body. Deterioration in the longitudinal arch may result in the flat foot.

The broad foot is thought to be caused by heredity, lack of exercise, and wearing shoes that do not fit properly. Fundamentally, it is caused by pes valgus or pes varus representing that the heel leans inside or outside. The pes valgus denotes the state of the foot having Achilles tendon leaning inward with respect to the ground from the view of the foot standing upright. The pes varus denotes the state of the foot having Achilles tendon leaning outward with respect to the ground. Generally, approximately 70% of the Japanese have a symptom of pes valgus.

The muscle is expanded and contracted under the load of body weight so as to be strengthened. As the foot kicking force is weakened, muscles of the sole and the lower limb may have difficulty in active expansion and contraction, resulting in degeneration. This may further deteriorate the aforementioned arches, leading to the broad foot and the flat foot.

Strengthening the muscle force of gripping toes is effective for alleviating the broad foot. The toe training method such as “towel catch” requiring catching of the towel with the sole with the aid of toes has been recommended. The effect derived from such method, however, is still restrictive.

The orthosis mainly for correcting or preventing the hallux valgus has been proposed in Patent dacuments listed below.

### CITATION LIST

### Patent Literature

### {Patent Literature 1} Japanese Patent Publication No. 5369321

### {Patent Literature 2} Japanese Patent Publication No. 4355364

### SUMMARY OF INVENTION

### Technical Problem

JP-5369321 and JP-4355364 disclose the orthosis intended to correct the toe suffered from the hallux valgus by forcibly pressing the subject toe with the aid of such fitting as the splint, the supporter or the taping.

The aforementioned orthosis as disclosed in JP-5369321 and JP-4355364 is configured to perform the correction by pressing down on toes so as not to move. This may cause the problem of deteriorating the foot muscle, thus weakening the foot kicking force (spring).

The ring-like or spectacle type ring-like orthosis has been proposed, which is designed to be fitted with the root of the toe. For certain users, such orthosis may excessively contract the root of the toe, thus causing the blood circulation disorders.

### SUMMARY

The present invention provides the foot health orthosis which naturally strengthen the foot kicking force (spring force) in walking to prevent deterioration in the muscle of foot and the part therearound, and is effective for preventing and correcting abnormality in the toe such as the floating toe, broad toe and hallux valgus for the purpose of promoting the health maintenance.

The present invention further provides the foot health orthosis, which is simply configured to be easily fitted, as well as naturally strengthen the foot kicking force (spring force) during walking with no excessive load imposed on the toe nor interference with walking.

### Solution to Problem

Embodiments configured to solve the aforementioned problem will be described referring to the corresponding drawings.

The present invention provides a foot health orthosis 1 to be fitted with a toe includes a flexible ring-like retainer 2 which can be fitted with the toe, and a cushion portion 3 formed on a part of the retainer 2, which is thicker than the retainer 2, and allowed to be held on a reverse surface of the toe in a fitted state.

The cushion portion 3 has a plate-like shape, which is thicker than the retainer 2, and made of an elastic member that exhibits elasticity in a top-reverse direction, for example, a gum material, a porous rubber material, or plastics.

The cushion portion 3 has a plate-like shape, which is thicker than the retainer 2, and made of a material that exhibits no elasticity, for example, plastics and a woody material.

The cushion portion 3 may be manufactured using a molded article of silicon and plastics.

The cushion portion 3 is formed on a part of the flexible ring-like retainer 2 made of a flexible band so as to connect both ends of the retainer 2.

The cushion portion 3 is formed on a part of the flexible ring-like retainer 2 while being folded to have opposite surfaces bonded so as to locate the part of the retainer 2 at an intermediate position.

The cushion portion 3 formed on a part of the flexible ring-like retainer 2 includes an upper section 3a and a lower section 3b constituting the cushion portion 3, opposite surfaces of which are bonded so that the part of the retainer 2 is located at an intermediate position.

The retainer 2 is made of a flexible ring-like flat belt. The cushion portion 3 with a thick plate-like shape, which is formed on a part of the retainer 2 has its top and reverse surfaces protruding from corresponding surfaces of the retainer 2, and its front and rear parts protruding from corresponding parts of the retainer 2.

For use, the foot health orthosis 1 according to the present invention is fitted with any toe of either left or right foot, and held with a retainer 2 while positioning a cushion portion 3 on the reverse surface of the toe.

If the cushion portion 3 is worn out or hardened owing to the long-time use, the adhesive tape may be wound around the outer periphery of the cushion portion 3 so as to be used. The adhesive tape wound around the outer periphery of the cushion portion 3 allows prolonged use and adjustment of the thickness.

The foot health orthosis 1 may be entirely subjected to the process of bacteria removal, antibacterial, and deodorization.

The walker having the foot health orthosis fitted with the foot allows the toe to be pushed up from the ground via the cushion portion 3 when landing during walking. When stepping forward, the toe is bent substantially in diagonally upward direction with respect to the foot so that the kicking force by the tiptoe is enhanced. This may strengthen or stimulate not only the toe muscle but also the muscle of the entire leg. In this way, the muscle may be stimulated easily with repetition along with the walking motion. This makes it possible to prevent deterioration in the muscle or to strengthen the muscle so as to promote health.

In the state where the foot health orthosis 1 is fitted with the toe, the thick cushion portion 3 is positioned on the reverse surface of the toe. This prevents the foot health orthosis 1 from digging into the outer periphery of the root of the toe. Therefore, the long-time use of the orthosis will not cause the risk of blood circulation disorder.

The present invention further provides a foot health orthosis 101 to be fitted with a toe, which includes a flexible ring-like retainer 2 which can be fitted with the toe, and an upper cushion portion 3A and a lower cushion portion 3B which are thicker than the retainer, formed on two opposite positions of the retainer 2, and allowed to be held on a reverse surface and a top surface of the toe in a fitted state.

A foot health orthosis 101 is fitted with the toe which has been deformed into substantially an inversed V-like shape called “hammer toe” as shown in FIG. 6 so that the deformed toe is corrected by pressing the subject toe from above with an upper cushion portion 3A in the shoe. The tip of the pressure pain point of the hammer toe is protected by the upper cushion portion 3A so that the pain is relieved.

### Advantageous Effects of Invention

The foot health orthosis according to the present invention is fitted with the toe so as to be pushed up from the ground via the cushion portion during walking. Then the force is applied to the toe to be bent substantially in diagonally upward direction with respect to the foot for enhancing the kicking force by the tiptoe. This may readily strengthen or stimulate the toe muscle, the foot muscle group and the lower limb muscle. The muscle is readily stimulated repeatedly for every step of walking.

As a result, formation of the foot arch is facilitated, or deterioration in formation of the arch is prevented, resulting in alleviation of the broad foot and the flat foot. Deterioration in the foot muscle and the leg muscle may be prevented to further promote health. This is also effective for preventing and correcting the abnormal toe, for example, the floating toe, the broad toe and the hallux valgus.

The foot health orthosis according to the present invention may be easily fitted with the toe by putting the ring-like retainer on the toe so that the kicking force by the tiptoe is naturally strengthened for every step of daily walking practice. This is significantly effective for alleviating abnormality in the foot such as the floating toe, the broad foot and the flat foot, without need of the toe training, for example, the towel catch.

The foot health orthosis according to the present invention has a simple structure with compact outer shape. It can be fitted with the toe even in the shoe without uneasy feel. It may be regularly fitted irrespective of indoor and outdoor environments without causing excessive deformation of the foot.

When the walker has the foot health orthosis fitted with the toe, the thick cushion portion is positioned on the reverse surface of the toe so as to prevent the ring-like retainer of the orthosis from digging into the outer periphery of the root of the toe. In spite of the long-time use of the orthosis, the risk of blood circulation disorder will not occur.

The cushion portion serves to protect the toe so as to prevent skin hardening, corn and callus of the toe.

Athlete's foot will be worsened by humidity, temperature, unsanitariness between toes. The foot health orthosis according to the present invention has the ring-like retainer positioned between adjacent toes, which is helpful in preventing Athlete's foot. The Athlete's foot prevention effect may be enhanced by impregnating the foot health orthosis with sterilization chemicals.

The size of the foot health orthosis may be variously and freely determined depending on the size of the user's toe to allow the use in accordance with circumstances. Additionally, it can be easily cleaned.

Another type of the orthosis is configured to have the upper and the lower cushion portions on two corresponding parts. This is effective for alleviating the toe deformed into the inversed V-like shape called the “hammer toe” as well as improving the kicking force by the toe. The upper cushion portion gives an effect of relieving the pain at the joint where the toe has been deformed.

### BRIEF DESCRIPTION OF DRAWINGS

FIG. 1A is a front view, FIG. 1B is a side view, and FIG. 1C is a perspective view of a foot health orthosis according to a first embodiment of the present invention;

FIGS. 2A and 2B are plan views, and FIG. 2C is a side view each representing an example of the use of the foot health orthosis;

FIGS. 3A to 3C represent examples of the size of the foot health orthosis;

FIGS. 4A to 4C are sectional views each representing the structure of a cushion portion of the foot health orthosis;

FIG. 5 is a front view of a foot health orthosis according to a second embodiment of the present invention;

FIG. 6 is a view representing the state where the foot health orthosis is in use; and

FIG. 7 is an explanatory view of arches formed on the sole both in lateral and longitudinal directions.

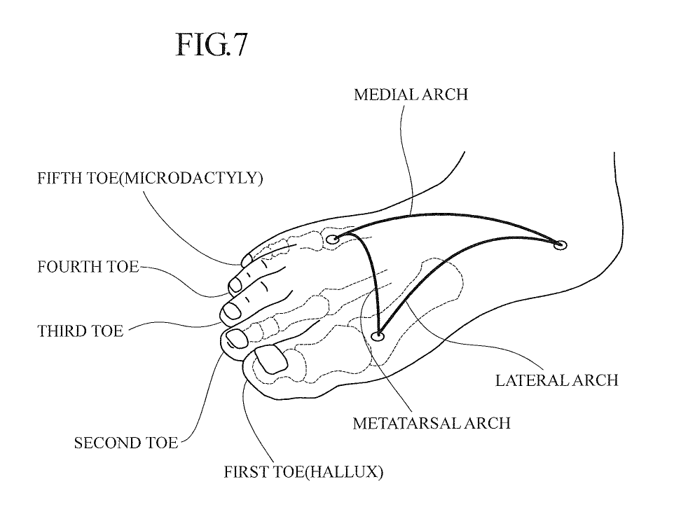

### DESCRIPTION OF EMBODIMENTS

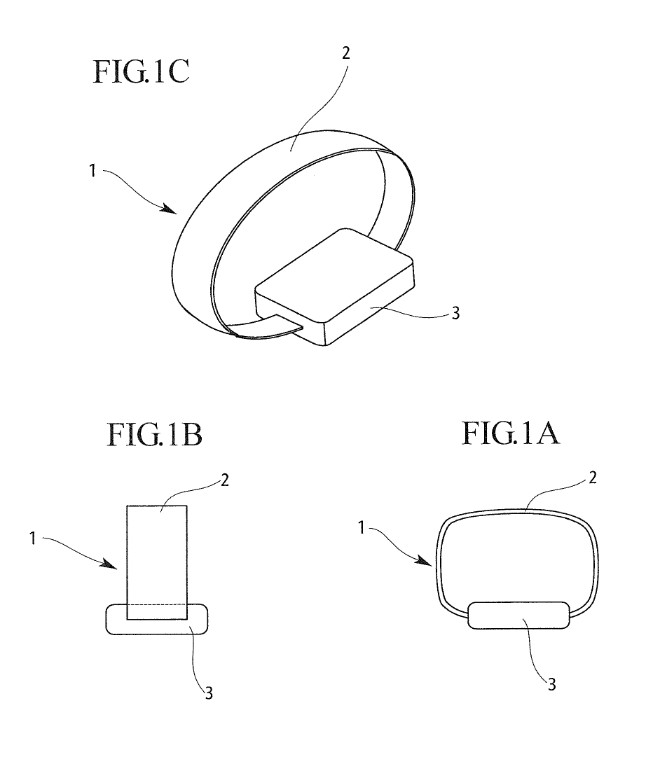

A foot health orthosis 1 according to a first embodiment of the present invention will be described referring to FIGS. 1A to 4C.

The foot health orthosis 1 according to the first embodiment includes a flexible ring-like retainer 2 which can be fitted with the toe, and a cushion portion 3 formed on a part of the retainer 2 and thicker than the retainer 2, which may be held on the reverse surface of the toe in the fitted state.

The cushion portion 3 has a plate-like structure with a thickness larger than that of the retainer 2, and made of the elastic member that exhibits elasticity in the top-reverse direction, including gum material, a porous rubber material such as relatively a hard sponge, and plastics. The cushion portion 3 is made of a relatively hard elastic material which is not easily deformed in the natural state where the orthosis is fitted and held with the toe.

The cushion portion 3 may also be made of the molded article of silicon and plastics, or the non-elastic material such as woody material.

The cushion portion 3 is formed into substantially a rectangular plate shape with small mattress-like structure as shown in the drawing. The cushion portion 3 may be formed into various shapes including elliptical, oval, and the one adapted to the reverse surface of the toe.

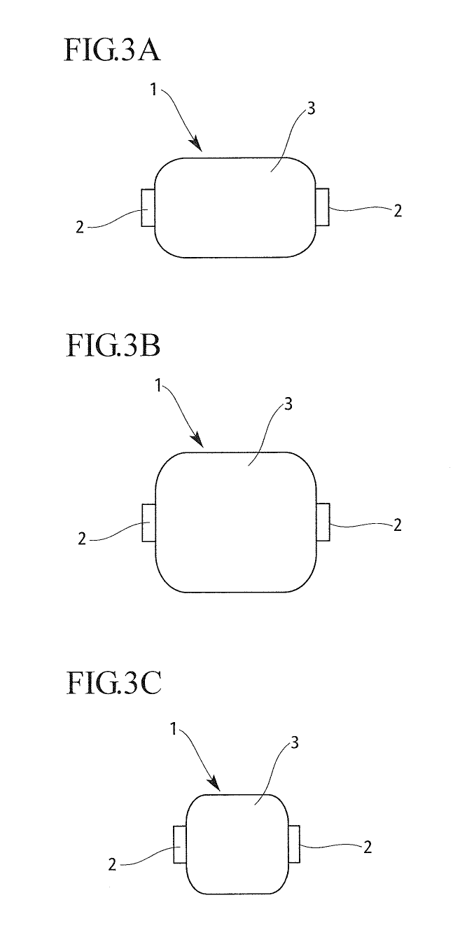

FIGS. 3A, 3B and 3C represent the respective sizes of a plurality of cushion portions 3 each with a small mattress structure. FIG. 3A illustrates the cushion portion 3 with 23 mm×15 mm, FIG. 3B illustrates the cushion portion 3 with 23 mm×20 mm, and FIG. 3C illustrates the cushion portion 3 with 15 mm×12 mm, respectively. The cushion portion 3 with the size suitable for the user's toe is selected and used. The cushion portions 3 with other sizes except those described above may be prepared for use.

The retainer 2 is made of a flexible ring-like flat belt, and continued inside the thickness of the cushion portion 3, which is located at the intermediate position. Then the cushion portion 3 has its top and reverse surfaces protruding from corresponding surfaces of the retainer 2, and its front and rear parts protruding from the corresponding parts of the retainer 2.

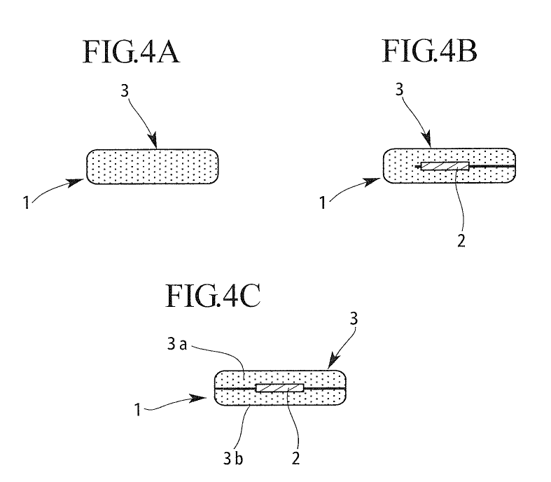

FIGS. 4A, 4B and 4C are sectional views each representing an example of the structure of the cushion portion 3.

Referring to the example shown in FIG. 4A, the cushion portion 3 is integrally molded to connect both ends of the retainer 2 made of the flexible band. Both ends of the retainer 2 may be fixed while being inserted into the cushion portion 3 at both ends. Both ends of the retainer 2 may be fixed while being abutted on the corresponding ends of the cushion portion 3 therein.

Referring to the example shown in FIG. 4B, the cushion portion 3 is folded back on top and reverse surfaces of the ring-like retainer 2 so that the folded surfaces are bonded to corresponding surfaces of the retainer.

Referring to the example shown in FIG. 4C, opposite surfaces of separately formed upper and lower sections 3a, 3b which constitute the cushion portion 3 are bonded to grip a part of the ring-like retainer 2.

For use, the foot health orthosis 1 is fitted with any toe of the left or right foot, which is held by the retainer 2 so that the cushion portion 3 is positioned on the reverse surface of the toe.

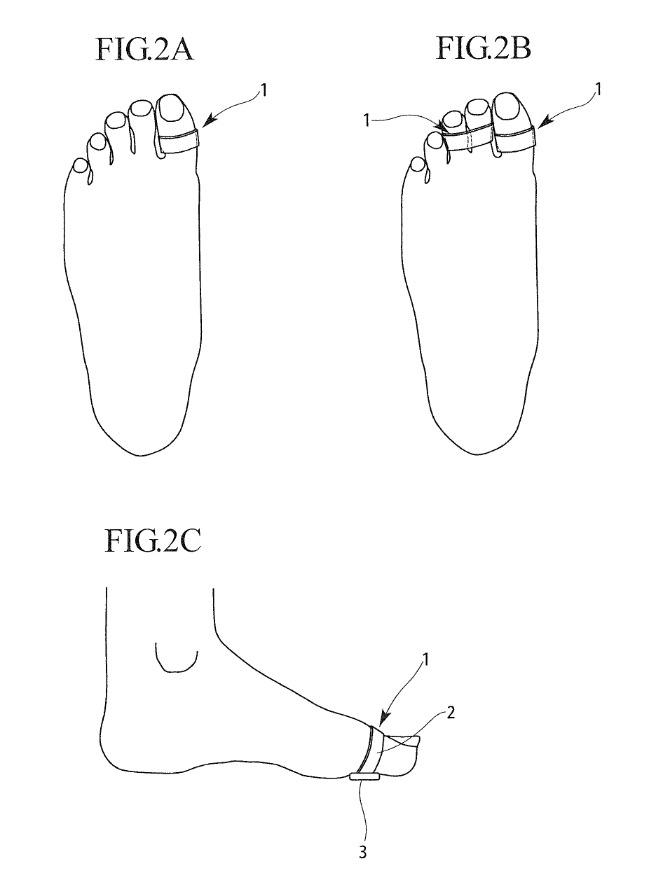

As FIG. 2A shows, the foot health orthosis may be fitted with each first toe (hallux) of the left and right feet. As FIG. 2B shows, the foot health orthosis 1 may be fitted with each first toe (hallux) of the left and right feet, and another one may be further fitted with the second and third toes of the foot together.

Fitting the foot health orthosis with the first toe (hallux) or the toe adjacent thereto of the user with a symptom of hallux valgus is effective for prevention or correction of such hallux valgus.

The foot health orthosis 1 may be fitted with any toe in accordance with the state of the toe so that the sole surface in contact with the ground is made in parallel with the ground in the stable state.

The toe of the user having the foot health orthosis 1 is pushed up from the ground via the cushion portion 3 subsequent to landing. The subject toe is bent substantially in diagonally upward direction with respect to the foot to enhance the kicking force by the tiptoe. This makes it possible to strengthen or stimulate not only the toe muscle but also the muscle of the entire foot.

The stimulation of the muscle is continuously applied for every step of walking without strain. This makes it possible to prevent deterioration in the muscle or to strengthen the muscle to promote the user's health.

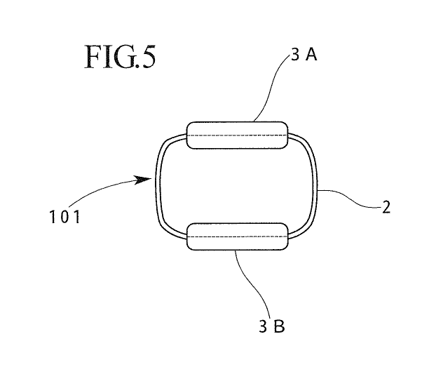

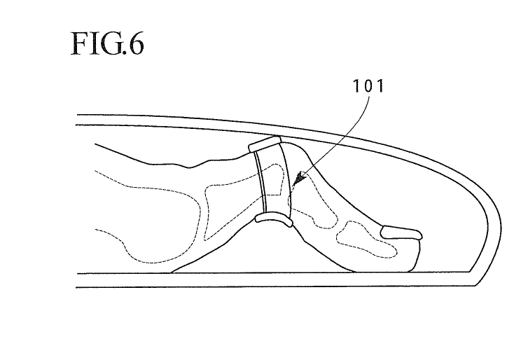

A foot health orthosis 101 according to a second embodiment of the present invention will be described referring to FIGS. 5 and 6.

The foot health orthosis 101 according to the second embodiment includes a flexible ring-like retainer 2 which can be fitted with the toe, and upper and lower cushion portions 3A, 3B which are disposed at two opposite parts of the retainer 2, and allowed to be held on the reverse and the top surfaces of the toe fitted with the foot health orthosis.

The foot health orthosis 101 is fitted with the toe which is deformed into substantially the inversed V-like shape, that is, the symptom of hammer toe. Specifically, it is used to allow correction of the subject toe in the shoe with the upper cushion portion 3A for applying pressure from above. In this case, it is preferable to fit the upper cushion portion 3A while being shifted backward with respect to the heel from the tip of the pressure pain point of the toe by approximately several millimeters. The upper cushion portion 3A functions to relieve the contact with or damage to the deformed joint part of the toe in the shoe. The function of the lower cushion portion 3B is similar to the one as described in the first embodiment.

### REFERENCE SIGNS LIST

1, 101 foot health orthosis
2, retainer
3, 3A, 3B cushion portion

1. A foot health orthosis which is fitted with a toe, comprising:
a flexible ring-like retainer which can be fitted with the toe; and
a cushion portion formed on a part of the retainer, which is thicker than the retainer, and allowed to be held on a reverse surface of the toe in a fitted state.

2. The foot health orthosis according to claim 1, wherein the cushion portion has a plate-like shape, which is thicker than the retainer, and made of an elastic member that exhibits elasticity in a top-reverse direction.

3. The foot health orthosis according to claim 1, wherein the cushion portion has a plate-like shape, which is thicker than the retainer, and made of a material that exhibits no elasticity.

4. The foot health orthosis according to claim 1, wherein the cushion portion is formed on a part of the flexible ring-like retainer made of a flexible band so as to connect both ends of the retainer.

5. The foot health orthosis according to claim 1, wherein the cushion portion is formed on a part of the flexible ring-like retainer while being folded to have opposite surfaces bonded so as to locate the part of the retainer at an intermediate position.

6. The foot health orthosis according to claim 1, wherein the cushion portion formed on a part of the flexible ring-like retainer includes an upper section and a lower section, opposite surfaces of which are bonded so that the part of the retainer is located at an intermediate position.

7. The foot health orthosis according to claim 1,
wherein the retainer is made of a flexible ring-like flat belt; and
the cushion portion with a thick plate-like shape which is formed on a part of the retainer has its top and reverse surfaces protruding from corresponding surfaces of the retainer, and its front and rear parts protruding from corresponding parts of the retainer.

8. A foot health orthosis which is fitted with a toe, comprising:
a flexible ring-like retainer which can be fitted with the toe; and
an upper cushion portion and a lower cushion portion which are thicker than the retainer, formed on two opposite positions of the retainer, and allowed to be held on a reverse surface and a top surface of the toe in a fitted state.
